(19) United States
(12) Patent Application Publication
(10) Pub. No.: US 2019/0333650 A1
(43) Pub. Date: Oct. 31, 2019

(54) DIAGNOSTIC IMAGE COLLABORATION

(71) Applicant: INTERNATIONAL BUSINESS MACHINES CORPORATION, Armonk, NY (US)

(72) Inventors: Terence Wilson, Newmarket (CA); Victoria Charlotte Dassen, Mississauga (CA); Jie Pei, Mississauga (CA)

(21) Appl. No.: 15/967,496

(22) Filed: Apr. 30, 2018

(51) Int. Cl. G16H 80/00; G06F 3/14; H04L 29/08; G16H 30/40; G16H 30/20

(52) U.S. Cl. CPC G16H 80/00; G06F 3/1454; H04L 67/10; G06F 17/241; G16H 30/40; G16H 30/20; H04L 67/02

(57) ABSTRACT

Methods and systems for collaborating on a medical image. One system includes an electronic processor. The electronic processor is configured to receive, from a first user device collaborating on the medical image as a presenter within a collaboration session for the medical image, presentation state information representing a current presentation state of the medical image as displayed on a display device of the first user device as modified by a user interaction of the presenter, and automatically transmit a presentation model based on the presentation state information to a second user device collaborating on the medical image as a collaborator, the second user device automatically modifying the medical image as displayed on a display device of the second user device based on the presentation model to mirror the user interaction with the medical image as displayed on the display device of the first user device.

### FIELD

Embodiments described herein relate to methods and systems for diagnostic image collaboration.

### SUMMARY

A picture archiving and communication system (“PACS”) is a central repository for various medical image studies of different modalities. A modality creates an image, an x-ray, a sonogram, a magnetic resonance imaging (“MRI”), and the like. There are defined workflows for reviewing and analyzing medial images, and a PACS server manages access to the medical images by other systems. A PACS viewer provides an interface for accessing medical images and provides various viewing options for one or more types of images. In some embodiments, the PACS viewer also include a dictation and speech-to-text mechanism that captures user audio input and converts the audio data to text data for insertion in a report, transmission to another system, or the like. Images awaiting review by a radiologist may be organized via worklists. Worklists are organizational structures that list the medical image studies a user is interested in reviewing and analyzing. For example, a radiologist may select a medical image study from a worklist, and the PACS viewer displays the medical images included within the selected medical image study. In some cases, the worklist is separate from the PACS viewer such that worklists are available for use with different viewers.

In some situations, multiple individuals collaborate on the treatment of a patient. For example, a radiologist or other specialist may be enlisted by a referring doctor to read medical images and provide the referring doctor with a report indicating the diagnosis of the patient's medical condition from the perspective of what is shown in the medical images. However, the report itself may not be sufficient to provide the referring doctor with the knowledge needed to adequately treat the patient. This generally results in further inquiries between the referring doctor and the radiologist.

Although online meetings and other collaboration systems exist, these systems may not be configured to handle medical image data. For example, in many collaboration systems, a screen shot is taken of a presenter's screen, which is then transmitted to other devices for display to other collaborators. Such screens may impair the diagnostic quality of medical images, which may hinder collaborators from properly reading the images and collaborating on a patient's health and treatment. Therefore, there is a need for a collaboration system that is directed to collaborating on medical imaging evaluations and that provides diagnostic quality imaging capabilities.

Accordingly, embodiments described herein provide a collaboration system that allows a presenter device to independently render diagnostic quality images while sharing presentation information for the images (in real-time or pseudo real-time) with other collaborator devices. The presentation information allows the collaborator devices to render the same images and apply the shared presentation information to display a medical image that mirrors the presentation on the presenter device.

For example, one embodiment provides a system for collaborating on medical image data captured as part of a medical imaging procedure. The system includes an electronic processor. The electronic processor is configured to receive, from a first user device collaborating on the medical image as a presenter within a collaboration session for the medical image, presentation state information representing a current presentation state of the medical image as displayed on a display device of the first user device as modified by a user interaction of the presenter, and automatically transmit a presentation model based on the presentation state information to a second user device collaborating on the medical image as a collaborator, the second user device automatically modifying the medical image as displayed on a display device of the second user device based on the presentation model to mirror the user interaction with the medical image as displayed on the display device of the first user device.

Another embodiment provides a method for collaborating on a medical image. The method includes displaying, with an electronic processor included in a first user device used by a presenter, the medical image on a display device of the first user device and transmitting an invitation to a collaborator via a second user device to join a collaboration session for the medical image. The method also includes, in response to receiving an acceptance of the invitation from the second user device, transmitting a presentation model to the second user device, the presentation model based on a current presentation state of the medical image as displayed on the display device of the first user device. In addition, the method includes, during the collaboration session, transmitting a subsequent presentation model to the second user device representing a subsequent presentation state of the medical image as displayed on the display device of the first user device as modified by the presenter using the first user device.

Yet another embodiment provides a non-transitory, computer-readable medium storing instructions that, when executed by an electronic processor, perform a set of functions. The set of functions includes displaying a medical image captured as part of a medical imaging procedure during a collaboration session on a display device of a collaborator device, receiving a presentation model for the medical image, the presentation model representing a current presentation state of the medical image as displayed on a display device of a presenter device as modified by a presenter, and automatically modifying the medical image as displayed on the display device of the collaborator device based on the presentation model to mirror the medical image as displayed on the display device of the presenter device.

Other aspects of the embodiments described herein will become apparent by consideration of the detailed description and accompanying drawings.

### BRIEF DESCRIPTION OF THE DRAWINGS

FIG. 1 schematically illustrates a system for providing diagnostic image collaboration according to some embodiments.

FIG. 2 is a user device included in the system of FIG. 1 according to some embodiments.

FIG. 3 is a flowchart illustrating a method for providing diagnostic image collaboration using the system of FIG. 1 according to some embodiments.

FIG. 4 is a screenshot of a user interface according to some embodiments.

FIGS. 5A and 5B are screenshots of a user interface according to some embodiments.

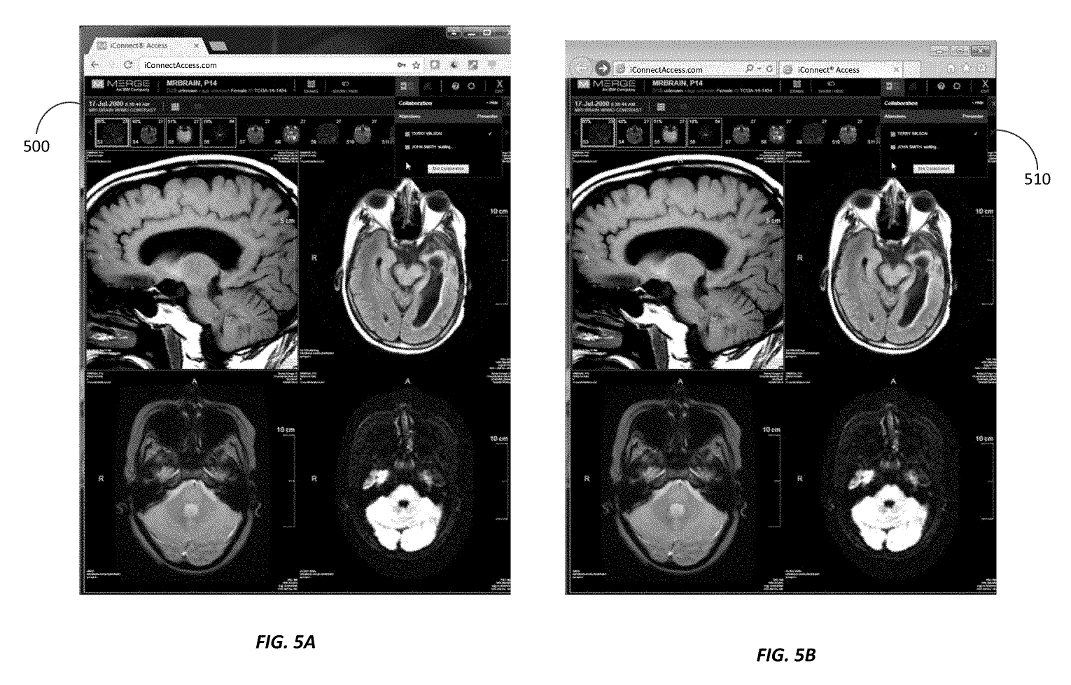

FIG. 6 is a screenshot of a user interface according to some embodiments

Other aspects of the embodiments described herein will become apparent by consideration of the detailed description.

### DETAILED DESCRIPTION

Before embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways.

Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and may include electrical connections or couplings, whether direct or indirect. Also, electronic communications and notifications may be performed using any known means including direct connections, wireless connections, and the like.

A plurality of hardware and software based devices, as well as a plurality of different structural components may be utilized to implement the embodiments described herein. In addition, embodiments described herein may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects of the embodiments described herein may be implemented in software (for example, stored on non-transitory computer-readable medium) executable by one or more processors. As such, it should be noted that a plurality of hardware and software based devices, as well as a plurality of different structural components, may be utilized to implement the embodiments described herein. For example, “mobile device,” “computing device,” and “server” as described in the specification may include one or more electronic processors, one or more memory modules including non-transitory computer-readable medium, one or more input/output interfaces, and various connections (for example, a system bus) connecting the components.

As described above, multiple individuals may collaborate on an evaluation of a patient based on imaging data. Typical collaboration systems, however, capture a screen shot of a presenter's screen and share the screen shot with the other collaborators. This type of collaboration can impact the quality of the imaging data displayed within the shared screen shot, which can impact a collaborator's contribution to the evaluation. Accordingly, embodiments described herein provide methods and systems for sharing presentation information from a presenter device to each collaborator device, wherein each collaborator device is configured to independently render a medical image and apply the shared presentation information to mirror the presenter's screen.

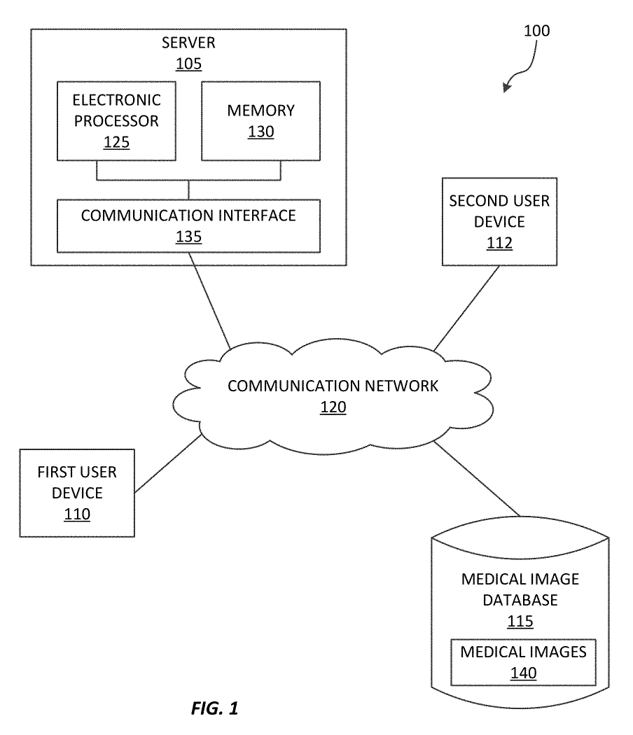

For example, FIG. 1 schematically illustrates a system 100 for providing diagnostic image collaboration according to some embodiments. The system 100 includes a server 105, a first user device 110, a second user device 112, and a medical image database 115. In some embodiments, the system 100 includes fewer, additional, or different components than illustrated in FIG. 1. For example, the system 100 may include multiple servers 105, medical image databases 115, or a combination thereof. Additionally, although the system 100 illustrated in FIG. 1 includes two user devices (for example, the first user device 110 and the second user device 112), it should be understood that the system 100 may include, for example, additional user devices, such as a third user device, a fourth user device, and the like.

The server 105, the first user device 110, the second user device 112, and the medical image database 115 communicate over one or more wired or wireless communication networks 120. Portions of the communication network 120 may be implemented using a wide area network, such as the Internet, a local area network, such as a Bluetooth™ network or Wi-Fi, and combinations or derivatives thereof Alternatively or in addition, in some embodiments, components of the system 100 communicate directly as compared to through the communication network 120. Also, in some embodiments, the components of the system 100 communicate through one or more intermediary devices not illustrated in FIG. 1.

The server 105 is a computing device that serves as a gateway for the medical image database 115. For example, in some embodiments, the server 105 is a PACS server. Alternatively, in some embodiments, the server 105 may be a server that communicates with a PACS server to access the medical image database 115. As illustrated in FIG. 1, the server 105 includes an electronic processor 125, a memory 130, and a communication interface 135. The electronic processor 125, the memory 130, and the communication interface 135 communicate wirelessly, over one or more communication lines or buses, or a combination thereof. The server 105 may include additional components than those illustrated in FIG. 1 in various configurations. The server 105 may also perform additional functionality other than the functionality described herein. Also, the functionality described herein as being performed by the server 105 may be distributed among multiple devices, such as multiple servers included in a cloud service environment. In addition, in some embodiments, the first user device 110, the second user device 112, or a combination thereof may be configured to perform all or a portion of the functionality described herein as being performed by the server 105.

The electronic processor 125 includes a microprocessor, an application-specific integrated circuit (ASIC), or another suitable electronic device for processing data. The memory 130 includes a non-transitory computer-readable medium, such as read-only memory (ROM), random access memory (RAM) (for example, dynamic RAM (DRAM), synchronous DRAM (SDRAM), and the like), electrically erasable programmable read-only memory (EEPROM), flash memory, a hard disk, a secure digital (SD) card, another suitable memory device, or a combination thereof. The electronic processor 125 is configured to access and execute computer-readable instructions (“software”) stored in the memory 130. The software may include firmware, one or more applications, program data, filters, rules, one or more program modules, and other executable instructions. For example, the software may include instructions and associated data for performing a set of functions, including the methods described herein.

The communication interface 135 allows the server 105 to communicate with devices external to the server 105. For example, as illustrated in FIG. 1, the server 105 may communicate with the first user device 110, the second user device, 112, the medical image database 115, or a combination thereof through the communication interface 135. In particular, the communication interface 135 may include a port for receiving a wired connection to an external device (for example, a universal serial bus (USB) cable and the like), a transceiver for establishing a wireless connection to an external device (for example, over one or more communication networks 120, such as the Internet, local area network (LAN), a wide area network (WAN), and the like), or a combination thereof.

The medical image database 115 stores a plurality of medical images 140 (collectively referred to as “the medical images 140” and individually referred to as “a medical image 140”). In some embodiments, the medical image database 115 is combined with the server 105, the first user device 110, the second user device 112, or a combination thereof. Alternatively or in addition, the medical images 140 may be stored within a plurality of databases, such as within a cloud service. Although not illustrated in FIG. 1, the medical image database 115 may include components similar to the server 105, such as an electronic processor, a memory, a communication interface, and the like. For example, the medical image database 115 may include a communication interface configured to communicate (for example, receive data and transmit data) over the communication network 120.

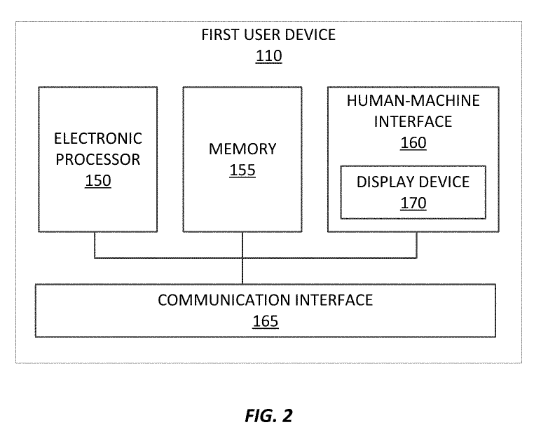

The first user device 110 and the second user device 112 are also computing devices and may include desktop computers, terminals, workstations, laptop computers, tablet computers, smart watches or other wearables, smart televisions or whiteboards, or the like. In some embodiments, the first user device 110 is located remotely from the second user device 112. FIG. 2 illustrates the first user device 110 included in the system 100 of FIG. 1. As illustrated in FIG. 2, the first user device 110 may include similar components as the server 105, such as an electronic processor 150, a memory 155, and a communication interface 165. As seen in FIG. 2, the first user device 110 also includes a human-machine interface 160 for interacting with a user. The human-machine interface 160 may include one or more input devices, one or more output devices, or a combination thereof. The human-machine interface 160 allows a user to interact with (for example, provide input to, receive output from, or a combination thereof) the first user device 110. For example, the human-machine interface 160 may include a keyboard, a cursor-control device (for example, a mouse), a touch screen, a scroll ball, a mechanical button, a display device (for example, a liquid crystal display (LCD)), a printer, a speaker, a microphone, or a combination thereof. As illustrated in FIG. 2, in some embodiments, the human-machine interface 160 includes a display device 170. The display device 170 may be included in the same housing as the first user device 110 or may communicate with the first user device 110 over one or more wired or wireless connections. For example, in some embodiments, the display device 170 is a touchscreen included in a laptop computer or a tablet computer. In other embodiments, the display device 170 is a monitor, a television, or a projector coupled to a terminal, desktop computer, or the like via one or more cables. Although not separately illustrated, it should be understood that the second user device 112 may include similar components and perform similar functions as the first user device 110 illustrated in FIG. 2. For example, the second user device 112 may also include an electronic processor, a memory, a communication interface, a human-machine interface, and the like.

A user may use the first user device 110 or the second user device 112 to access and view one or more medical images 140 and interact with a medical image 140. For example, the user may access a medical image 140 stored in the medical image database 115 (through a browser application or a dedicated application stored on the first user device 110 that communicates with the server 105) and view the medical images 140 on the display device 170 associated with the first user device 110. The user may also interact with the medical images 140 using the human-machine interface 160 of the first user device 110. For example, a user may modify content of a medical image 140 (for example, by drawing on a medical image 140), modify a display property of a medical image 140 (for example, by modifying a contrast property, a brightness property, and the like), or a combination thereof.

As noted above, the first user device 110 and the second user device 112 may be used to collaboratively evaluate or diagnose a patient based on one or more medical images 140. For example, a radiologist (for example, a first user) may use the first user device 110 to collaborate with a physician (for example, a second user) using the second user device 112. In particular, the radiologist may annotate the medical image 140 to identify a portion of the medical image 140 in which the radiologist is basing a diagnostic opinion on. As described in more detail below, the radiologist's interaction with the medical image 140 may be simultaneously displayed to the medical image 140 as displayed to the physician (for example, in real time or pseudo real time). As used in the present application, “real-time” means receiving data, processing the data, and returning results of processing the data without significant delay. For example, “real-time” may include processing data within seconds or milliseconds so that results of the processing are available virtually immediately.

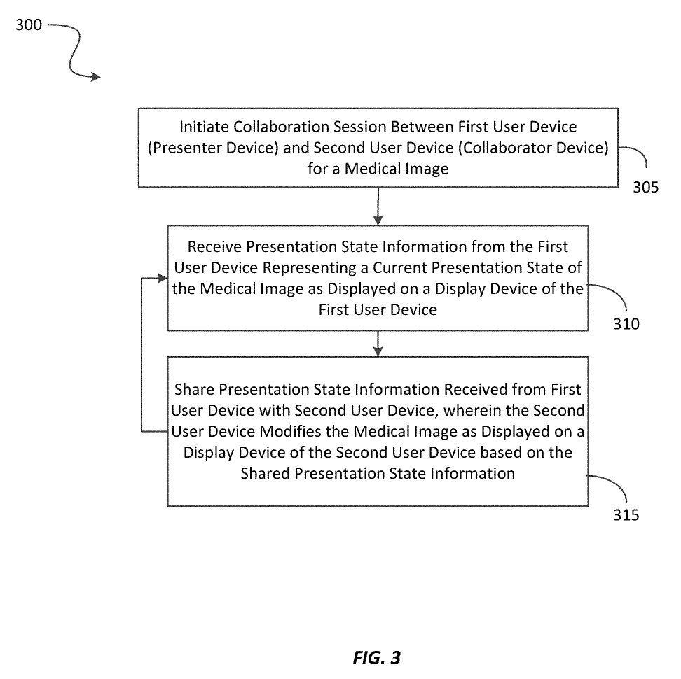

FIG. 3 is a flowchart illustrating a method 300 for providing diagnostic image collaboration according to some embodiments. The method 300 is described here as being performed by the server 105 (the electronic processor 125 executing instructions). However, as noted above, the functionality performed by the server 105 (or a portion thereof) may be performed by other devices, including, for example, the first user device 110, the second user device 112, or a combination thereof (via, for example, the electronic processor 150 executing instructions). For example, as described herein, the server 105 may be configured to receive presentation state information for a medical image as displayed at a user device and forward the presentation state information to other user devices collaborating on the same medical image. However, in other embodiments, a user device acting as the presenter for a collaboration session for a medical image may forward presentation state information (and updates thereof) directly (not through the server 105) to the other user devices collaborating on the medical image.

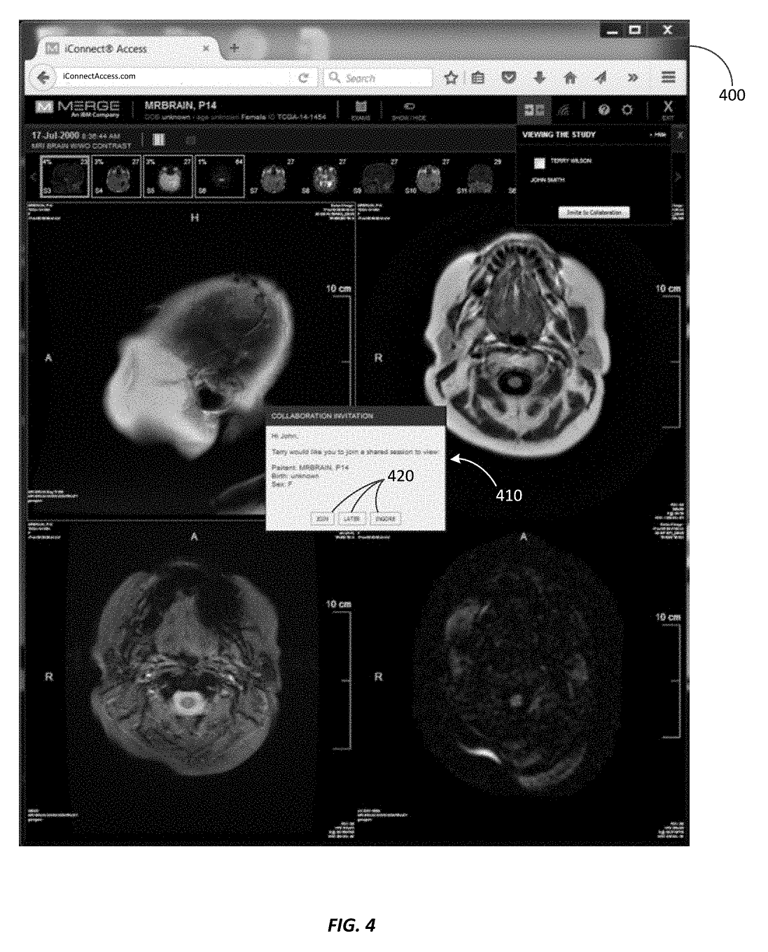

As illustrated in FIG. 3, the method 300 includes initiating a collaboration session between the first user device 110 and the second user device 112 (at block 305). In some embodiments, a user of the first user device 110 or the second user device 112 may initiate the collaboration session with the server 105 and the user may then invite other users (participants) to the collaboration session. For example, for purposes of the description of FIG. 3 and as one example, assume the user of the first user device 110 desires to initiate a collaboration session for an image study locally stored on the first user device 110. In this situation, the user (hereinafter referred to as the presenter) opens the study containing a plurality of medical images and (optionally) interacts with at least one of the medical images 140, such as by changing a presentation state (zoom level, contrast, and the like), adding an annotation, or the like. At this point, the presenter can initiate a collaboration session by sending a request to the server 105. The server 105 may generate a new collaboration session with a unique identifier and share the unique identifier with the presenter, which the presenter can share with other users to invite those users to the collaboration session. For example, the presenter may be able to generate an invitation including the identifier and send the invitation to other users (for example, in an e-mail message, a text message, an instant message, a pop-up window, or the like). Alternatively or in addition, the presenters may designate users to be invited to the collaboration session to the server 105, and the server 105 may generate and transmit invitations to these users. FIG. 4 illustrates a user interface 400 displayed on the second user device 112 that includes a dialogue box 410 prompting the user of the second user device 112 (hereinafter referred to as the “first collaborator”) to join the collaboration session. In some embodiments, the dialogue box 410 includes information associated with the collaboration session. For example, the dialogue box 410 may identify a patient, a medical image study, one or more medial images 140, a patient characteristic, and the like. The first collaborator can use the information included in the dialogue box to manually open the study included in the collaboration session. As illustrated in FIG. 4, the dialogue box 410 also includes a plurality of buttons 420. The first collaborator selects one of the plurality of buttons 420 to accept or decline the invitation. In response to selecting an accept button from the plurality of buttons 420, the device used by the first collaborator may transmit an identifier of the device, the collaborator, or both to the server 105, which the server 105 may use to track the participants of the collaboration session. If the second user device 112 does not already have a copy of the medical images 140 associated with the collaboration session, accepting the invitation to join the session may also automatically download a copy of the associated images 140 to the second user device 112.

In some embodiments, the electronic processor 125 initiates the collaboration session in response to receiving the request from the presenter. Alternatively or in addition, the electronic processor 125 may initiate the collaboration session in response to the first collaborator accepting the invitation to join the collaboration session. In some embodiments, the collaboration session is a web-based collaboration session. It should be understood that more than two users may participate in a collaboration session and users may join a session at different times. For example, continuing with the example scenario set forth above, the presenter may also invite a user of a third user device (hereinafter referred to as the “second collaborator”) to join the collaboration session at the same time the first collaborator is invited or at a later time.

Returning to FIG. 3, after the collaboration session is initiated (for example, after the first collaborator joins the session), the server 105 receives presentation state information from the first user device 110 (used by the presenter) representing a current presentation state of the medical image as displayed on a display device of the first user device 110 (used by the presenter) (at block 310) and shares this presentation state information with the second user device 112 (used by the first collaborator) (at block 315). For example, the server 105 may be configured to generate a presentation model based on the received presentation state information and transmit the presentation model to each user included in the session. The presentation model represents the current presentation state of the medical image as displayed by the presenter's device. In some embodiments, the presentation model includes one or more properties of the displayed medical image 140 that were modified by the presenter as represented in the received presentation state information.

Accordingly, after joining the collaboration session, the second user device 112 (used by the first collaborator) receives the presentation model from the server 105 and uses the received presentation model to modify the same medical image as displayed on the second user device 112 to match the medical image as displayed on the first user device 110. For example, the second user device 112 may update the presentation state of the medical image as displayed on the second user device 112 to the settings included in the presentation model. Thus, the medical image 140 displayed on the second user device 112 is automatically modified to mirror the medical image 140 as displayed on the first user device 110 without impacting a quality of the medical image 140 as displayed on each collaborator. In particular, by rendering the medical image 140 independently at each collaborator device (as compared to merely sharing a screen shot), diagnostic image quality is preserved. It should be understood that, in some embodiments, the second user device 112 is configured to apply the presentation model to the medical image 140. However, in other embodiments, a separate device, such as the server 105, may be configured to apply the presentation model to the medical image 140 and transmit a modified version of the medical image 140 to the second user device 112 for display. In other words, embodiments described herein may be used with client-side rendering of medical images and server-side rendering of medical images. As noted above, in some embodiments, only one user included in a collaboration session is designated as the presenter, and a user that is not designated as a presenter is thus restricted from interacting or otherwise modifying the displayed medical image.

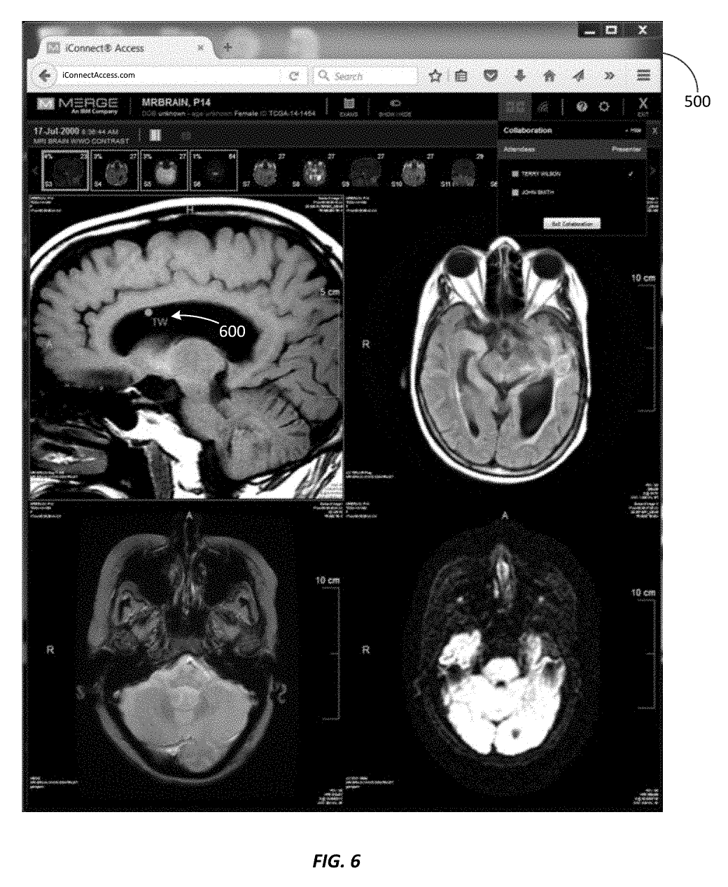

While the collaboration session is still active, the server 105 continues to receive presentation state information from the presenter's device and transmits this presentation state information to the device used by each collaborator to maintain the medical image 140 as displayed to each collaborator synchronized (in real-time or pseudo real-time) with the medical image as displayed to the presenter. The presenter device may be configured to transmit presentation state information periodically, in response to a change in the presentation state of the medical image as displayed by the presenter's device (such as based on the presenter's interaction with the displayed image), or a combination thereof. For example, if the presenter selects a zoom button X times to increase the zoom level of the medical image, the device used by the presenter transmits presentation state information to the server 105 indicating the new presentation state of the medical image (for example, the zoom level for the image has changed to X %). A presenter may interact with a displayed medical image in various ways. For example, as one example, a presenter may interact with a medical image 140 by annotating the medical image, such as by identifying a particular portion of the medical image 140. A presenter may also interact with a displayed medical image 140 by modifying the brightness property, contrast property, or other display property of the image, modifying a size of the image or a zoom level of the image, or the like. In some embodiments, the presenter's cursor position may also be displayed within the medical image 140. For example, as illustrated in FIG. 6, an identifier 600 displayed within the medical image 140 may designate the current position of the presenter's cursor. In some embodiments, the current cursor position of other participants (non-presenters) may also be displayed within the medical image 140, which allows the other participants to point out an area or location within the image 140, make a suggestion, or the like.

It should be understood that the presentation state information transmitted to the server 105 by the presenter device may take various forms. For example, as described above, the presentation state information may include presentation control commands that describe the effect of the presenter's interactions with a displayed medical image 140. In particular, the presentation state information can represent the current display state of the image 140 on the presenter's device, including, for example, a zoom level, a brightness level, a layout or configuration applied by the viewer, cursor position coordinates, and the like. However, alternatively or in addition, the actual user interactions with the medical image 140 (mouse clicks, mouse movements, key presses, touches, and the like) are transmitted to the server 105 as the presentation state information. The server 105 may forward these interactions to the collaborator devices, which can effectively replay the interactions on the medical image 140 as displayed on each collaborator device. Alternatively or in addition, the server 105 may convert these received interactions into presentation state information, which the server 105 can forward to each collaborator (for example, included in a presentation model), use to generate a modified version of the medical image 140 for transmission to each collaborator device, or a combination thereof. Also, in some embodiments, the presentation model transmitted by the server 105 may include the presentation state information as received from the presenter device (unaltered). Thus, in these situations, the server 105 may act as a relay for the presentation state information transmitted by the presenter device.

Embodiments described herein provide a collaboration system that preserves diagnostic image quality by sharing image modifications between participants and allowing the shared modifications to be applied to the medical image displayed at each participant as compared to capturing and comparing screen shots. In other words, each participant in the collaboration session (collaboration endpoint) renders its own version of a medical image while maintaining synchronization of the medical images displayed to each participant based on the medical image rendered by the presenter of the session. Not only does this configuration preserve the quality of the medical image, but may reduce the amount of data shared between the participant device as only modifications needs to be shared and not screen shots.

Various features and advantages of the embodiments described herein are set forth in the following claims.

What is claimed is:

1. A system for collaborating on medical image data captured as part of a medical imaging procedure, the system comprising:
an electronic processor configured to
receive, from a first user device collaborating on the medical image as a presenter within a collaboration session for the medical image, presentation state information representing a current presentation state of the medical image as displayed on a display device of the first user device as modified by a user interaction of the presenter, and
automatically transmit a presentation model based on the presentation state information to a second user device collaborating on the medical image as a collaborator, the second user device automatically modifying the medical image as displayed on a display device of the second user device based on the presentation model to mirror the user interaction with the medical image as displayed on the display device of the first user device.

2. The system of claim 1, wherein the user interaction modifies a display property of the medical image displayed on the display device of the first user device.

3. The system of claim 2, wherein the second user device automatically modifies the medical image displayed on the display device of the second user device by modifying a display property of the medical image displayed on the display device of the second user device to match the display property of the medical image displayed on the display device of the first user device as modified by the user interaction.

4. The system of claim 1, wherein the user interaction adds an annotation to the medical image as displayed on the display device of the first user device.

5. The system of claim 4, wherein the second user device automatically modifies the medical image displayed on the display device of the second user device by adding the annotation to the medical image as displayed by the display device of the second user device.

6. The system of claim 1, wherein the collaboration session is a web-based collaboration session.

7. The system of claim 1, wherein the electronic processor is further configured to receive a request from the first user device to initiate the collaboration session between the first user device and the second user device.

8. The system of claim 7, wherein the electronic processor is further configured to prompt the second user device to join the collaboration session.

9. The system of claim 1, wherein the presentation model includes a property of the medical image displayed on the display device of the first user device as modified by a user of the first user device.

10. A method for collaborating on a medical image, the method comprising:
displaying, with an electronic processor included in a first user device used by a presenter, the medical image on a display device of the first user device;
transmitting an invitation to a collaborator via a second user device to join a collaboration session for the medical image;
in response to receiving an acceptance of the invitation from the second user device, transmitting a presentation model to the second user device, the presentation model based on a current presentation state of the medical image as displayed on the display device of the first user device; and
during the collaboration session, transmitting a subsequent presentation model to the second user device representing a subsequent presentation state of the medical image as displayed on the display device of the first user device as modified by the presenter using the first user device.

11. The method of claim 10, further comprising receiving the presentation model at the second user device and applying the presentation model to the medical image as displayed on a second display of the second user device to mirror the medical image as displayed on the display device of the first user device.

12. The method of claim 10, wherein transmitting the subsequent presentation model to the second user device includes transmitting presentation state information from the first user device to a server, generating the subsequent presentation model with the server based on the presentation state information received from the first user device, and transmitting the presentation model from the server to the second user device.

13. The method of claim 10, wherein transmitting the subsequent presentation model to the second user device includes transmitting the subsequent presentation model to the second user device periodically.

14. The method of claim 10, wherein transmitting the subsequent presentation model to the second user device includes the subsequent presentation model to the second user device in response to a modification of the medical image by the presenter.

15. A non-transitory, computer-readable medium storing instructions that, when executed by an electronic processor, perform a set of functions, the set of functions comprising:
displaying a medical image captured as part of a medical imaging procedure during a collaboration session on a display device of a collaborator device;
receiving a presentation model for the medical image, the presentation model representing a current presentation state of the medical image as displayed on a display device of a presenter device as modified by a presenter; and
automatically modifying the medical image as displayed on the display device of the collaborator device based on the presentation model to mirror the medical image as displayed on the display device of the presenter device.

16. The computer-readable medium of claim 15, wherein the current presentation state of the medical image includes a display property of the medical image as displayed on the display device of the presenter device.

17. The computer-readable medium of claim 15, wherein the current presentation state of the medical image includes an annotation added to the medical image as displayed on the display device of the presenter device.

18. The computer-readable medium of claim 15, wherein receiving the presentation model includes receiving the presentation model from a server communicating with the presenter device to receive the current presentation state.

19. The computer-readable medium of claim 15, wherein receiving the presentation model includes receiving the presentation model from the presenter device.

20. The computer-readable medium of claim 15, wherein automatically modifying the medical image as displayed on the display device of the collaborator device based on the presentation model includes automatically setting a display property of the medical image as displayed on the display device of the collaborator device to a value included in the presentation model.
